(19) United States
(12) Patent Application Publication
(10) Pub. No.: US 2011/0118561 A1
(43) Pub. Date: May 19, 2011

(54) REMOTE CONTROL FOR A MEDICAL MONITORING DEVICE

(75) Inventors: Abdolreza Yaghoobzadeh Tari, Tehran (IR); Hamid Azizzadeh, Tehran (IR); Mahmood Reza Merati, Tegran (IR)

(73) Assignee: MASIMO Corporation, Irvine, CA (US)

(21) Appl. No.: 12/618,682

(22) Filed: Nov. 13, 2009

(51) Int. Cl. A61B 5/00

(52) U.S. Cl. 600/301

(57) ABSTRACT

A physiological monitoring system, according to embodiments of the disclosure, can independently control multiple displays to provide displays of measured physiological parameters than can differ from each other in format and/or selected parameters. Individual display monitors can be customized to display the parameters of interest to a particular medical professional more prominently. In order to facilitate controlling multiple displays, a controller in communication with the physiological monitoring system can be attached or positioned near a user of a display. The controller can remotely change the display output from the physiological monitoring system. The controller can be attached to a particular display and control the corresponding output for that display. Typically, commands from the controller affect only the display output for the particular display and not the display output for other displays.

### FIELD OF THE DISCLOSURE

The present disclosure relates to medical sensors and specifically to patient monitoring systems.

### BACKGROUND OF THE DISCLOSURE

Patient monitoring of various physiological parameters of a patient is important to a wide range of medical applications. Oximetry is one of the techniques that has developed to accomplish the monitoring of some of these physiological characteristics. It was developed to study and to measure, among other things, the oxygen status of blood. Pulse oximetry—a noninvasive, widely accepted form of oximetry—relies on a sensor attached externally to a patient to output signals indicative of various physiological parameters, such as a patient's constituents and/or analytes, including for example a percent value for arterial oxygen saturation, carbon monoxide saturation, methemoglobin saturation, fractional saturations, total hematocrit, billirubins, perfusion quality, or the like. A pulse oximetry system generally includes a patient monitor, a communications medium such as a cable, and/or a physiological sensor having light emitters and a detector, such as one or more LEDs and a photodetector. The sensor is attached to a tissue site, such as a finger, toe, ear lobe, nose, hand, foot, or other site having pulsatile blood flow which can be penetrated by light from the emitters. The detector is responsive to the emitted light after attenuation by pulsatile blood flowing in the tissue site. The detector outputs a detector signal to the monitor over the communication medium, which processes the signal to provide a numerical readout of physiological parameters such as oxygen saturation (SpO2) and/or pulse rate.

High fidelity pulse oximeters capable of reading through motion induced noise are disclosed in U.S. Pat. Nos. 6,770,028, 6,658,276, 6,157,850, 6,002,952 5,769,785, and 5,758,644, which are assigned to Masimo Corporation of Irvine, Calif. (“Masimo Corp.”) and are incorporated by reference herein. Advanced physiological monitoring systems can incorporate pulse oximetry in addition to advanced features for the calculation and display of other blood parameters, such as carboxyhemoglobin (HbCO), methemoglobin (HbMet), total hemoglobin (Hbt), total Hematocrit (Hct), oxygen concentrations, glucose concentrations, blood pressure, electrocardiogram data, temperature, and/or respiratory rate as a few examples. Typically, the physiological monitoring system provides a numerical readout of and/or waveform of the measured parameter. Advanced physiological monitors and multiple wavelength optical sensors capable of measuring parameters in addition to SpO2, such as HbCO, HbMet and/or Hbt are described in at least U.S. patent application Ser. No. 11/367,013, filed Mar. 1, 2006, titled Multiple Wavelength Sensor Emitters and U.S. patent application Ser. No. 11/366,208, filed Mar. 1, 2006, titled Noninvasive Multi-Parameter Patient Monitor, assigned to Masimo Laboratories, Inc. and incorporated by reference herein. Further, noninvasive blood parameter monitors and optical sensors including Rainbow™ adhesive and reusable sensors and RAD-57™ and Radical-7™ monitors capable of measuring SpO2, pulse rate, perfusion index (PI), signal quality (SiQ), pulse variability index (PVI), HbCO and/or HbMet, among other parameters, are also commercially available from Masimo Corp.

To facilitate monitoring of patients at remote locations or during patient transport, portable, battery-operated, patient monitors capable of independent operation are currently available. Further, docking systems capable of mechanically accepting and electrically connecting to portable patient monitors have been developed and are commercially available from Masimo Corp. Such systems allow a patient transported by ambulance to a hospital emergency room to be monitored during the trip using a portable patient monitor, and when the patient is delivered to the emergency room, the portable patient monitor can be docked to a docking system having monitoring peripherals in the emergency room, such as an additional display. The use of the docking system eliminates the need to remove and replace the existing sensors monitoring the patient. A portable device and docking system are described at least in U.S. Pat. No. 7,530,949 issued May 12, 2009, titled Dual-Mode Pulse Oximeter, assigned to Masimo Corp., and incorporated by reference herein.

### SUMMARY OF THE DISCLOSURE

Docking systems allow the expansion of the capabilities of a portable patient monitor by providing access to additional peripherals. One application for a docking system is to provide multiple displays for displaying the measured parameters of a patient monitor. In emergency or operating rooms, there are typically a team of nurses and doctors treating a patient. The members of the team can be located in different locations with different available viewing angles such that the use of multiple displays is beneficial.

It is therefore desirable to provide a physiological monitoring system having multiple display devices. The multiple displays can be driven by a single patient monitor, typically of a portable design. By having a single patient monitor and multiple displays, a single set of sensors can be used to monitor the physiological parameters while providing more access to the monitoring information.

Further, controls for independently altering the display of each of the multiple displays can be provided such that the output on each display can be customized depending on the requirements of the viewer. For example, different members of an operating team are likely to focus on particular parameters based on their function on the team and individually customizable displays can enhance the effectiveness of medical professionals.

A physiological monitoring system, according to embodiments of the disclosure, can independently control multiple displays to provide displays of measured physiological parameters that can differ from each other in format and/or selected parameters. Individual display monitors can be customized to display the parameters of interest to a particular medical professional more prominently. In order to facilitate controlling multiple displays, a controller in communication with the physiological monitoring system can be attached or positioned near a user of a display. The controller can remotely change the display output from the physiological monitoring system. The controller can be attached to a particular display and control the corresponding output for that display. Typically, commands from the controller affect only the display output for the particular display and not the display output for other displays.

### BRIEF DESCRIPTION OF THE DRAWINGS

FIG. 1 illustrates a block diagram of a physiological monitoring system having an external display and remote station;

FIG. 2 illustrates an embodiment of the remote station of FIG. 1;

FIG. 3 illustrates a cross-section of the remote station of FIG. 2 taken along line 3;

FIGS. 4A and 4B illustrate the engagement of the mount and remote control of the remote station of FIG. 2;

FIG. 4C illustrates an embodiment of a physiological monitoring system;

FIG. 5 illustrates an embodiment of the base station having a built-in display;

FIG. 6 illustrates the base station of FIG. 5 with a docked patient monitor;

FIG. 7 illustrates an alternative embodiment of the base station of FIG. 1;

FIG. 8 illustrates the base station of FIG. 7 having a docked patient monitor;

FIG. 9 illustrates an embodiment of the base station connected to an external display;

FIG. 10 illustrates a block diagram of an embodiment of the patient monitor and the base station of FIG. 7;

FIG. 11 illustrates a block diagram of an embodiment the base station of FIG. 5.

### DETAILED DESCRIPTION OF THE PREFERRED EMBODIMENT

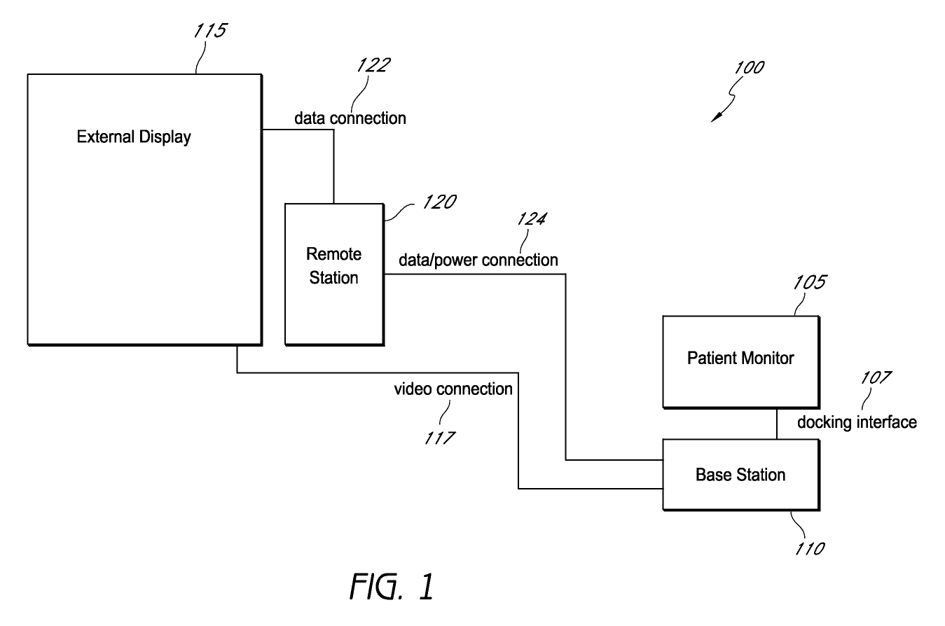

FIG. 1 illustrates a patient monitoring system 100, such as for pulse oximetry, having an external display and remote station. The patient or physiological monitoring system includes a patient monitor 105 having a primary display, a base station 110, one or more external displays devices, 115 and/or one or more controllers or remote stations 120. The patient monitor can be docked to the base station and electronically connected through a docking interface 107. The base station can be connected to an external display 115 via a communications medium 117, such as a video cable, which carries an output signal from the base station. The video cable can comprise a Video Graphics Array (VGA), High-Definition multimedia interface (HDMI), Digital Video Interface (DVI), DisplayPort and/or similar cable interface. Typically, the external display screen is larger than the patient monitor's primary display. The external display can be connected to a remote station 120 via an optional communications medium 122, such as a data cable. In an embodiment, the external display is a touch screen monitor and the communications medium 122 provides the input from operation of the touch screen to the remote station 120. The remote station can be connected to the base station 110 via a communication medium 124 and/or power line 124. The remote station can send data to and/or receive data from the base station. In one embodiment, the communication medium 117 for the video signal from the base station connects to the remote station, which relays the video signal to one or more external displays. In one embodiment, one or more of the communications mediums 117, 122, 124 can be wireless connections.

The patient monitor 105 can be a portable device capable of independent operation from the patient monitor system 100 in a first configuration. In one embodiment, the patient monitor 105 comprises at least one processor, a memory, a primary display and an internal power source, such as, preferably, a rechargeable battery. The primary display is preferably an LCD and can be a touch screen display. Various sensors can be attached to the patient monitor for monitoring physiological parameters, such as pulse oximetry sensors. For example, operating in the first configuration, the patient monitor can be used in an ambulance to provide monitoring of patients that are being transported to the hospital. Using its primary display, the patient monitor can display monitored parameters. Once the patient reaches the hospital, the patient monitor can operate in a second configuration where it can be docked to the base station 110 to form a patient monitoring system. In such a configuration, the patient monitor can transmit a display to the larger external display 115 and/or receive power from the base station.

Docking can include mechanically attaching the patient monitor to a base station and/or forming an electrical connection between the patient monitor 105 and the base station 110. The electrical connection or docking interface 107 can allow power and/or data to transmit between the patient monitor and the base system in either direction. For example, the display output from the patient monitor can be transmitted to the base station while remote commands from the external display 115 and/or remote station 120 can be transmitted to the patient monitor. The use of a patient monitor and docking system advantageously allows continuous monitoring of the patient throughout the patient's transport and arrival.

The base station 110 can provide power and/or data connectivity to a docked or connected patient monitor 105. For example, the base station can provide a wired or wireless network connection and/or connections to additional peripherals, such as one or more external displays 115, one or more remote station 120, and/or the like through one or more outputs. The base station can have a display output, such as a VGA, HDMI, DVI and/or the like, which transmits an output signal to the base station. The output signal includes the values of the physiological parameters monitored by the patient monitor. The output signal from the base station can originate from the patient monitor and be transmitted to the base station. In an embodiment, the base station 110 comprises a built-in display for displaying patient monitor data from the patient monitor.

The external display 115 receives an electronic signal from the base station 110 comprising patient monitor data. In one embodiment, the external display displays additional information to that shown by the patient monitor's display, such as additional measured parameters, additional waveforms, and/or more detail about measured parameters. The external display can be a touch screen monitor, allowing a user, such as medical professional, to select which parameters to monitor or how to display information on the screen.

The remote station 120 connects to the patient monitor 105 and can control the patient monitor, including its output to the external display 115. The remote station 120 can be connected directly to the patient monitor or through the base station. The remote station can attach to the external display, allowing a user to control the output of the display from a position remote from the patient monitor. In an embodiment, the remote station comprises a remote control and a mount. The mount attaches to the external display. The remote control attaches to the mount but can be detached and operated away from the mount.

The remote station 120 can have a plurality of inputs and/or outputs. Inputs can include power and/or a data inputs from the base station 110 and/or external display 115. Outputs can include a data output, such as for commands, to the base station 110 or patient monitor 105. For example, the external display 115 can be a touch screen monitor providing user inputs from the touch screen interface to the remote station. The user inputs can be transmitted to the remote station through a touch screen cable 122 or wirelessly. In response to the received commands, the remote station can transmit those commands to the base station and/or patient monitor. The commands can direct the patient monitor to change displayed parameters, display additional waveforms, cycle through available parameters or waveforms, change display formats, start or stop monitoring, display a menu, record data, activate an alarm, mute audio, and/or the like. In some embodiments, the data cable 122 can be unnecessary, such as when the external display is not a touch screen monitor.

In some embodiment, the components of the system can be connected wirelessly or by a combination of wired and wireless connections. For example, the output signal from the base station 110 can be transmitted wirelessly to the external display 115, such as by Wireless Home Digital Interface (WHDI), WirelessHD, and/or the like. In one embodiment, the remote station 120 can serve as a wireless bridge between wired external displays and the base station. The output signal from the base station can be transmitted wirelessly to the remote station. The remote station can be connected by a cable to the external display and can convert the wireless signal to a wired signal for output to the connected external display.

In some embodiments, the base station 110 provides multiple output signals for multiple external displays. A remote station 120 can be attached to each external display and assigned to control a particular output to a particular display, such that multiple remote stations can operate in the same room without interfering with each other. For example, a first remote station can be assigned to control a first output signal from the base station while a second remote station can be assigned to control a second output signal. The first and second remote station can be associated with a first and second display respectively. A command to the first remote station can cause the first display to change independently of the second display.

In some embodiments, the capabilities of the base station are integrated into the patient monitor and a separate base station is unnecessary. For example, the patient monitor can have a wireless connection to the other components of the patient monitoring system. By using wireless connections, a patient monitor can operate portably and independently when away from the other components but can connect to other components by using a wireless discovery process, well-known in the art, once in range. In some embodiments, the patient monitor is configured for stationary use only and no docking station is used.

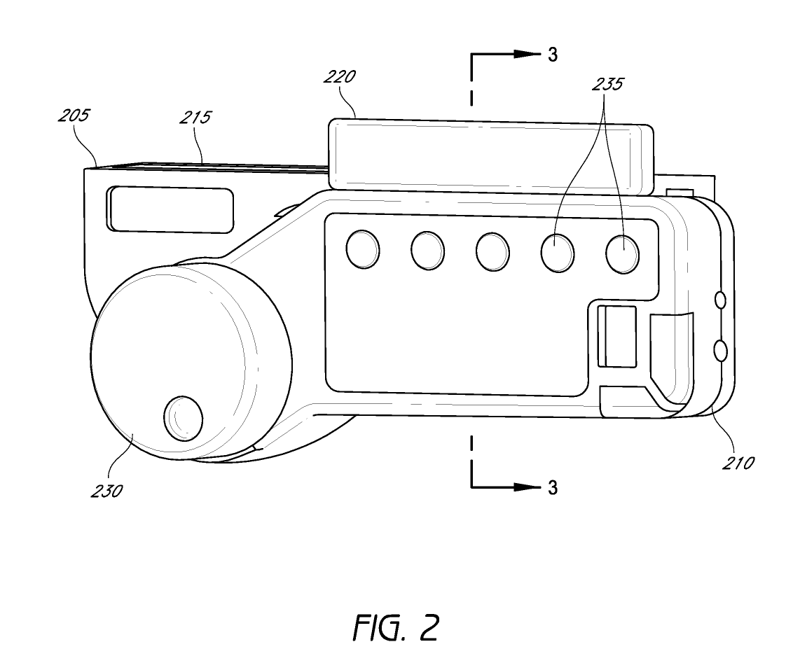

FIG. 2 illustrates an embodiment of the remote station of FIG. 1. In the illustrated embodiment, the remote station 120 comprises a mount 205 and a remote control 210. The mount 205 attaches to a first surface along an attachment surface 215. Attachment can be through adhesive, Velcro, mounting screws, clamp(s) and/or the like. In one embodiment, a corner attachment 220 of the mount provides a placement guide and/or second attachment point to a second surface generally perpendicular to the first surface, such as a corner of a display.

In one embodiment, the remote control 210 is a hand-sized generally rectangular housing containing electrical component within. The electrical components can include one or more processors, memory, a transmitter and/or receiver. The electrical components are configured to transmit and/or receive data to and from other components, such as the external display 115, base station 110, and/or patient monitor 105 through a communications medium. The communication medium can be a cable into the housing or a wireless connection, such as infrared, radio, Bluetooth, and/or the like. The remote control can have an internal power source, such as a battery, or an external power source, such as power line to an electrical outlet or another component of the patient monitoring system.

The remote control 210 is releasably attached to the mount 205 through at least one connector. The remote control comprises an input knob 230 and a plurality of input buttons 235 for inputting commands, such as those disclosed above. The input knob can be rotated and/or depressed. For example, rotating the input knob can cause the display to scroll through display options in a menu and depressing the knob selects a menu item. Alternatively, rotation of the knob can cause the display to change between display options. In one embodiment, the input knob can select between characters on a virtual keyboard and depressed to select a character.

In an embodiment, the remote control 210 can be connected by a cable to the external display and/or the base station. The remote control 210 can contain a wireless receiver, such as an infrared receiver, for receiving commands from a wireless controller (not shown).

In one embodiment, the mount 205 can house electrical components. The mount can include electric components for receiving or transmitting a signal from the patient monitor 105, base station 110, and/or external display 115. The mount can further include a wireless transmitter and/or receiver for communicating wirelessly with the remote control 210. For example, the mount can be connected by wire to the base station 110 and wirelessly to the remote control 210, transmitting commands entered on the remote control 210 to the base station 110.

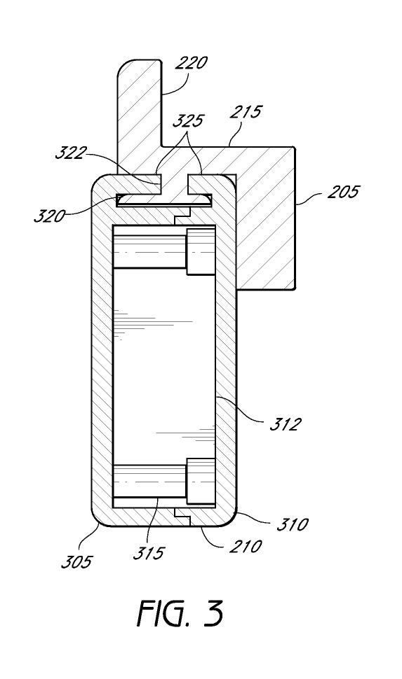

FIG. 3 illustrates a cross-section of the remote station of FIG. 2 taken along line 3. The mount 205 can be attached to a display monitor or other object along attachment surfaces 220, 215. The remote control 210 comprises a front housing 305 and a rear housing 310 forming an enclosure 312 for housing electrical components. The front housing and rear housing are connected by at least one connector 315, such as a column for receiving screws. The remote control can be connected to the mount 205 through an attachment mechanism. In one embodiment, the attachment mechanism comprises an attachment tab 320 formed perpendicularly to and extending outwardly from a support column 322, which together define at least one groove extending longitudinally along the mount, the at least one groove aligning with one or more attachment arms 325 formed on the remote control. In turn, the attachment arms 325 form a slot for the attachment tab 320. The attachment arms allow the remote control to be slidably attached to the mount. In one embodiment, the attachment mechanism components can be switched, with the attachment arms on the mount and the attachment tab on the remote control.

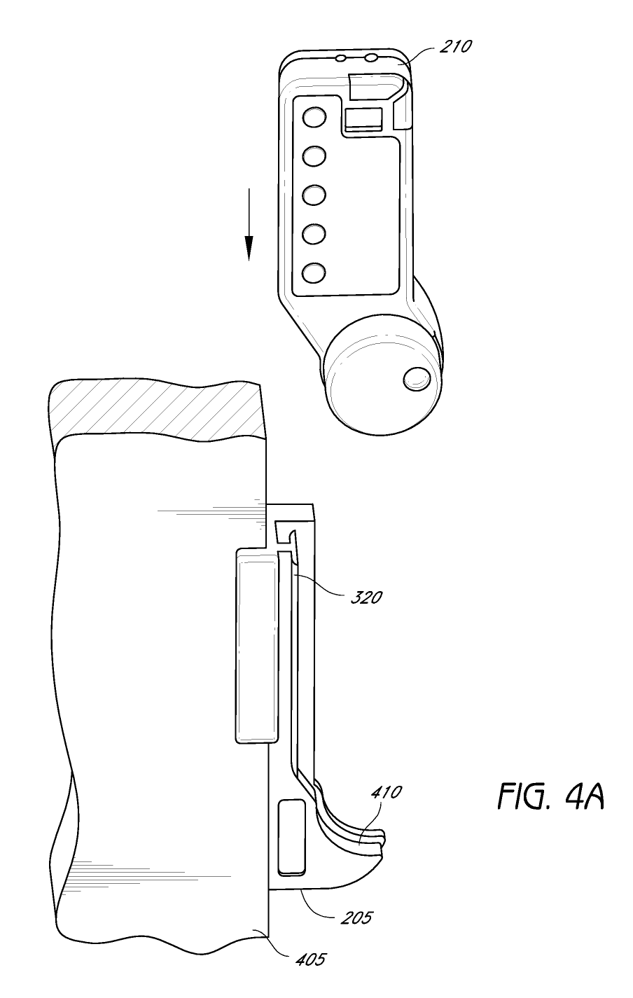

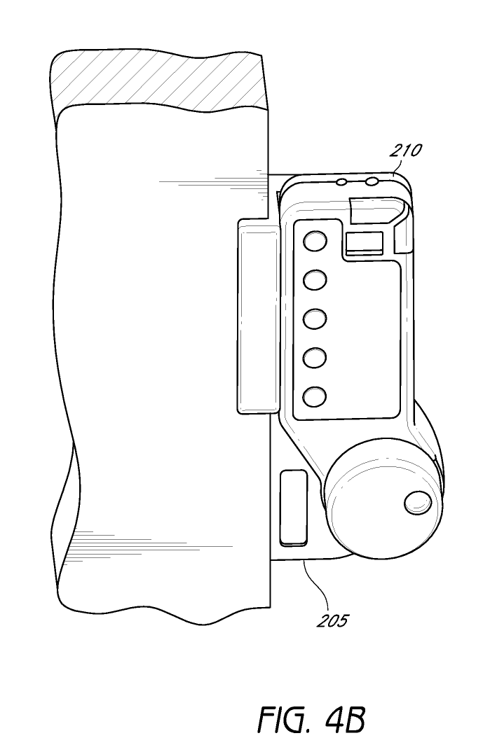

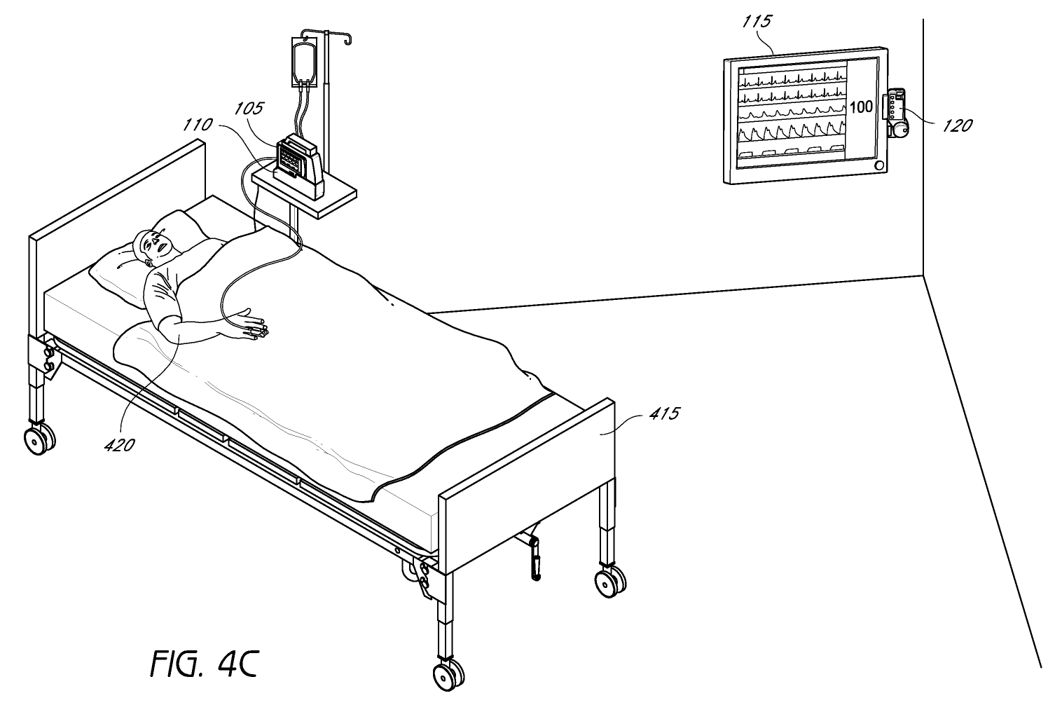

FIGS. 4A and 4B illustrate the engagement of the mount and remote control of the remote station of FIG. 2. In FIG. 4A, the mount 205 is attached to a corner surface 405, such as the corner of a display. The remote control 210 slides into the mount 205 from above by aligning the attachment tab 320 on the mount with the slot on the remote control. A stop or cradle 410 prevents further downward movement of the remote control with respect to the mount. The remote control can be removed from the mount by sliding the remote control up until the attachment arms disengage from the attachment tab 320.

FIG. 4B illustrates the remote control 210 engaged with the mount 205. The remote control can be stored on the mount when not in use. The physical proximity of the remote control to the display monitor allows a medical professional to quickly change the displayed output on the monitor.

In some embodiments, the remote control 210 can be attached to the mount 205 using adhesive, Velcro, and/or other releasable connection. In one embodiment, the remote control 210 does not use a mount.

FIG. 4C illustrates an embodiment of a physiological monitoring system. A hospital room contains a hospital bed 415 and a patient 420. A patient monitor 105 attached to a base station 110 is positioned alongside the bed. One or more sensors connected to the patient monitor are monitoring various physiological parameters, which are displayed on the patient monitor. An external display 115 provides a second display of the physiological parameters in another part of the hospital room. Attached to the display is a remote station 120 allowing a user to control the display output of the external display while positioned away from the patient monitor. Connections between the remote station, patient monitor, base station, and/or external monitor can be through wired or wireless connections.

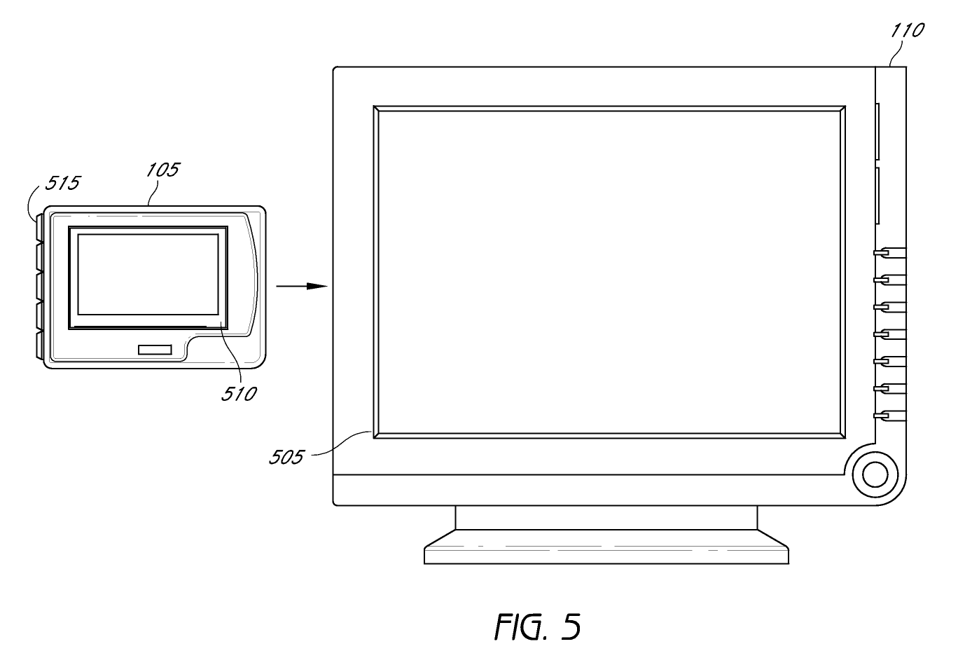

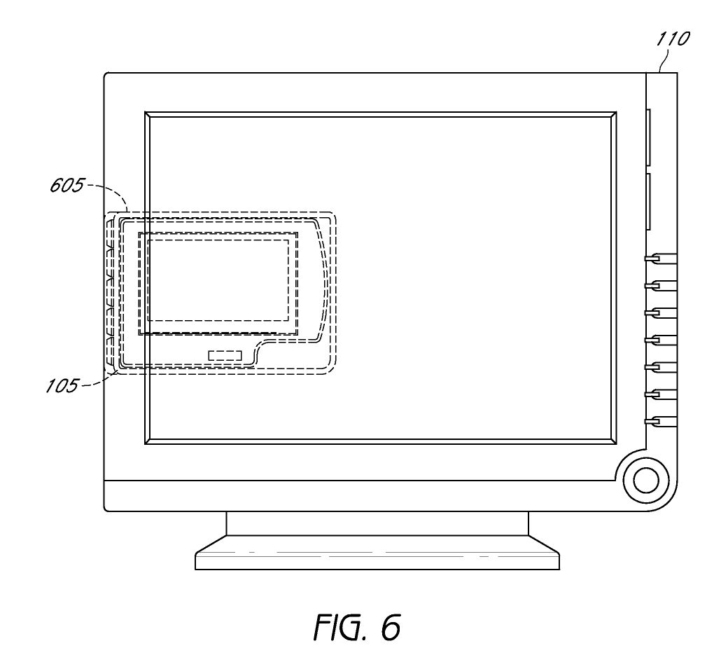

FIG. 5 and FIG. 6 illustrate an embodiment of the base station having a built-in display. In FIG. 5, the patient monitor 105 can dock within the base station 110. The base station 110 includes a built-in display 505, which can be a touch screen display. The built-in display 505 can mirror the information on the primary display 510 or display additional information, such as additional values of monitored parameters. The sensor connectors 515 on the patient monitor are left accessible externally so that sensors can be attached or detached while the patient monitor is docked.

FIG. 6 illustrates an embodiment of the patient monitor 105 docked within the base station 110. The base station comprises a docking recess 605 shaped to fit the patient monitor 105. The patient monitor 105 can form an electrical connection with the base station 110 through a docking interface (not shown). The connection allows power and/or data to flow between the base station and the patient monitor.

In one embodiment, the base station 110 further comprises at least one video output for transmitting an output signal to an external display 115. The external display 115 can mirror the base station's display 505 or display data independently. Providing multiple displays allows the patient to be monitored from different positions in the room or by multiple medical professionals, such as by different members of a surgical team.

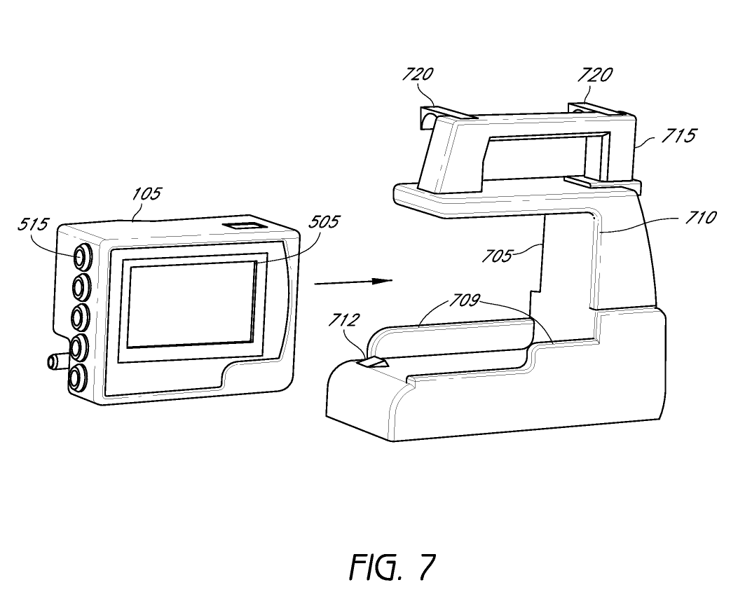

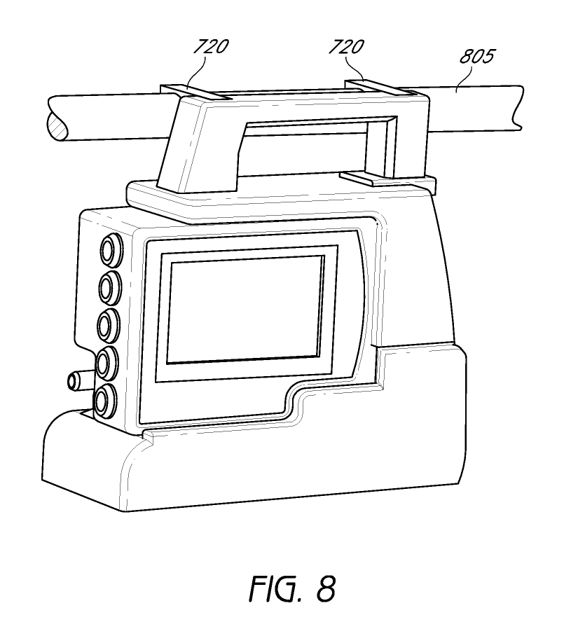

FIG. 7 and FIG. 8 illustrate another embodiment of the base station of FIG. 1. In the embodiment of FIG. 7, the base station 110 is a light-weight docking station for the patient monitor 105. The base station includes a recess 705 with dimensions that conform to the patient monitor. The recess is defined on three sides by the base station, with portions of the base station forming a top, a side, and a bottom of the recess. One or more rails 709 formed on the bottom portion of the base station secure the patient monitor within the recess. The recess includes an opening 710 over the primary display 505 of the patient monitor, allowing a user to view the display. The sensor connections 515 of the patient monitor are left exposed when docked to allow attachment and/or detachment of sensors. A locking mechanism 712, such as spring biased protrusion, locks the patient monitor into the base station by fitting within a corresponding recess (not shown) on the patient monitor. A release mechanism (not shown) can be actuated to release the locking mechanism and allow the patient monitor to be removed.

The base station 110 can house electronic components within itself. These components can provide additional connectivity and functionality, such as monitoring of additional physiological parameters, network connectivity, display outputs, and/or a power connection. The base station can include a handle 715 for carrying the base station. The base station can further include one or more mounting hooks 720 for attachment of the base station to a headboard/footboard, side rail, roll stand and IV stand, bed frame, and/or the like. The base station can include a battery and/or a removable power cord and can be transportable, allowing extended portable operation of the patient monitor 105.

FIG. 8 illustrates the base station of FIG. 7 with the patient monitor inserted into the recess. The mounting hooks 720 allow the base station to be attached to, for example, a horizontal surface or bar 805.

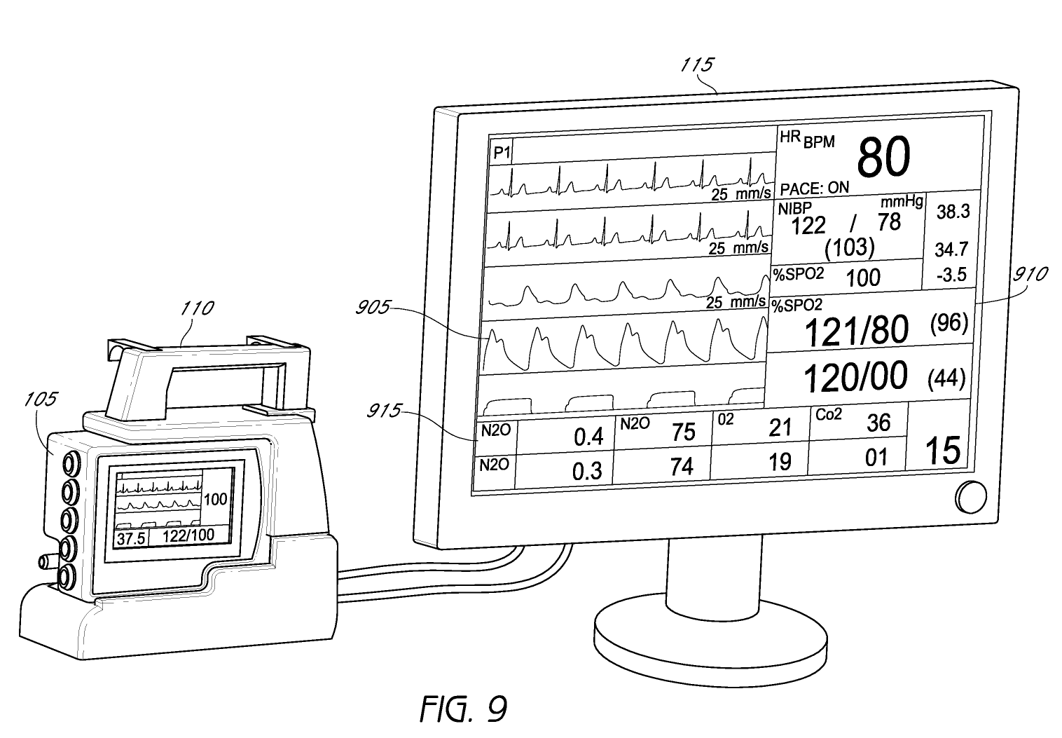

FIG. 9 illustrates the base station of FIG. 7 connected to an external display 115. Typically, the external display is larger than the primary display 505 of the patient monitor 105. The larger size advantageously allows additional information to be displayed on the external display, such as additional waveforms 905, additional detail about existing parameters 910 and/or additional parameters 915. The display area of the external display can be used to display a numerical value and/or waveforms of parameters such as heart rate, blood pressure, SpO2, N2O, O2, CO2, and/or the like.

In some embodiments, the external display 115 mirrors or depicts the same information as the patient monitor 105. The external display can also depict the same physiological parameters but in an alternate format. For example, a parameter value can be displayed using larger font sizes, displayed over time using a waveform, and/or displayed more prominently, such as by using different colors, placement or highlighting. In some embodiments, additional parameters or more detail about an existing parameter can be displayed to provide more information about the patient's condition. The information on the display can be changed based on commands received from the remote station 120 from the user.

In some embodiments, multiple external displays 115 can be attached to the base station 110. The base station can have multiple display outputs for each external display 115. Alternatively, external displays can be daisy chained together using a single display output on the base station 110. The output signal can be the same for each monitor or each monitor can receive its own output signal. DisplayPort, a packet-based display interface, is one example of a technology allowing multiple output signals using a single display output.

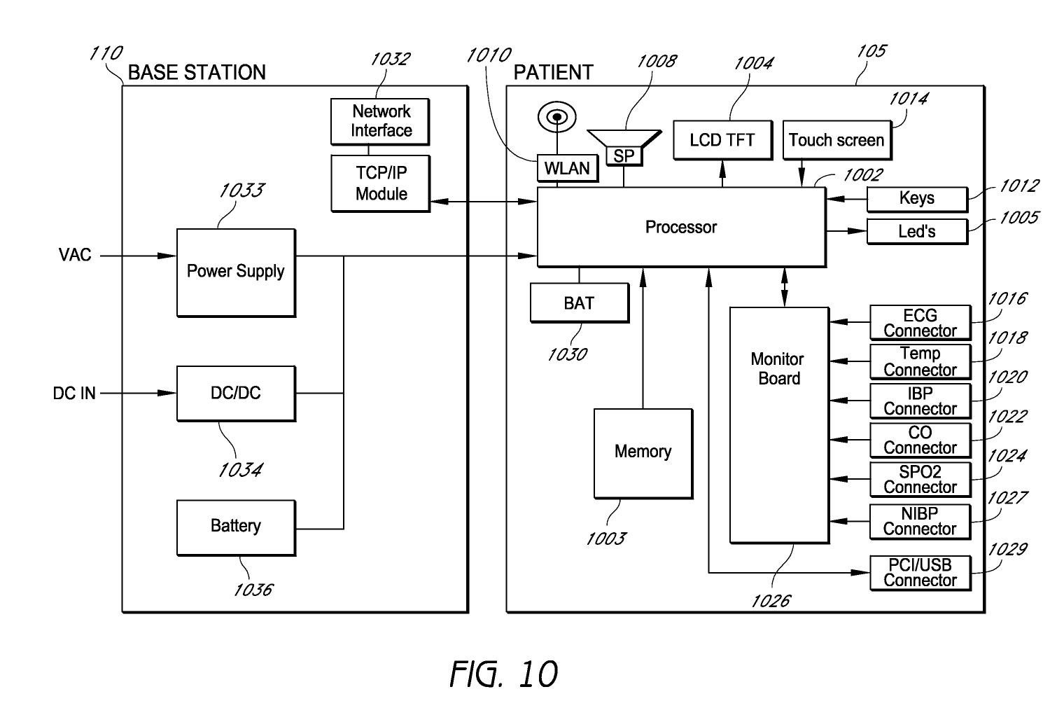

FIG. 10 illustrates a block diagram of an embodiment of the patient monitor 105 and the base station 110. In one embodiment, the patient monitor includes at least one processor 1002, memory 1003, such as non-volatile, volatile and/or solid state memory, a display 1004, LED's 1005, a speaker 1008, and a wireless receiver and/or transmitter 1010 for connection to a network.

The processor can receive user inputs from keys 1012 or a touch screen sensor 1014. The memory can store information such as boot data, manufacturing serial numbers, diagnostic failure history, adult SpO2 and pulse rate alarm limits, neonate SpO2, pulse rate alarm limits, SpO2, pulse rate trend data, program data, and/or the like. The display can be monochrome or color, and preferably is an LCD. The LED's can provide an indication of the status of the patient monitor and/or the patient. The speaker can provide an alarm signal in response to detection of patient parameters indicating a medical emergency. A monitoring board 1026 measures and/or analyzes the inputs from one or more sensors attached to one or more connectors 1016, 1018, 1020, 1022, 1024, 1027 on the patient monitor, such as sensors for ECG, temperature, carbon dioxide (CO2), invasive and/or noninvasive blood pressure (IBP, NIBP), SpO2, respiration, multi-gas an and/or any other physiological parameter measurement sensors and transmits the information to the processor. In one embodiment, the monitoring board is a Masimo Rainbow SET® OEM board, such as the MX-3 board. Additional sensors connectors and monitoring boards can be included, such as a monitoring board for non-invasive blood pressure (NIPS). Moreover, a single general processor can perform all of the functionality of the patient monitor or multiple processors can be used to perform the various processing tasks. A serial connection 1029, such as a peripheral component interconnect (PCI) or universal serial bus (USB), allows connection of external peripheral devices. Power can be provided to the monitor from an internal power source 1030, such as a rechargeable battery.

The processor and/or monitoring board can store and analyze the acquired data. In particular, the processor and/or monitoring board can run algorithms for analyzing the acquired data. The central processing system controls the transfer of data to the display panel for display and to the LAN via either a hardwired or wireless connection.

The base station 110 components include a network interface 1032, such as an Ethernet port, a power supply 1033 and/or optional battery 1036. The network interface can include a TCP/IP module and allows the patient monitoring system 100 to connect to computer systems on the hospital's network, such as a central database for storing patient information. In one embodiment, the power supply can accept a range of voltage, such as 100-220 VAC at 50/60 Hz and convert the voltage for internal use, such as to 220V/5.6V. A DC/DC converter 1034 allows the base station to receive power from a DC power source, such as a 10-14 VDC source, and convert the voltage for internal use, such as to 5.6V. A battery charger can charge the internal power source 1030 of the patient monitor. The base station can connect to the patient monitor 105 through a connection interface. The interface allows data and/or power to flow between base station and patient monitor. One or more display outputs (not shown) provide display information to one or more external displays.

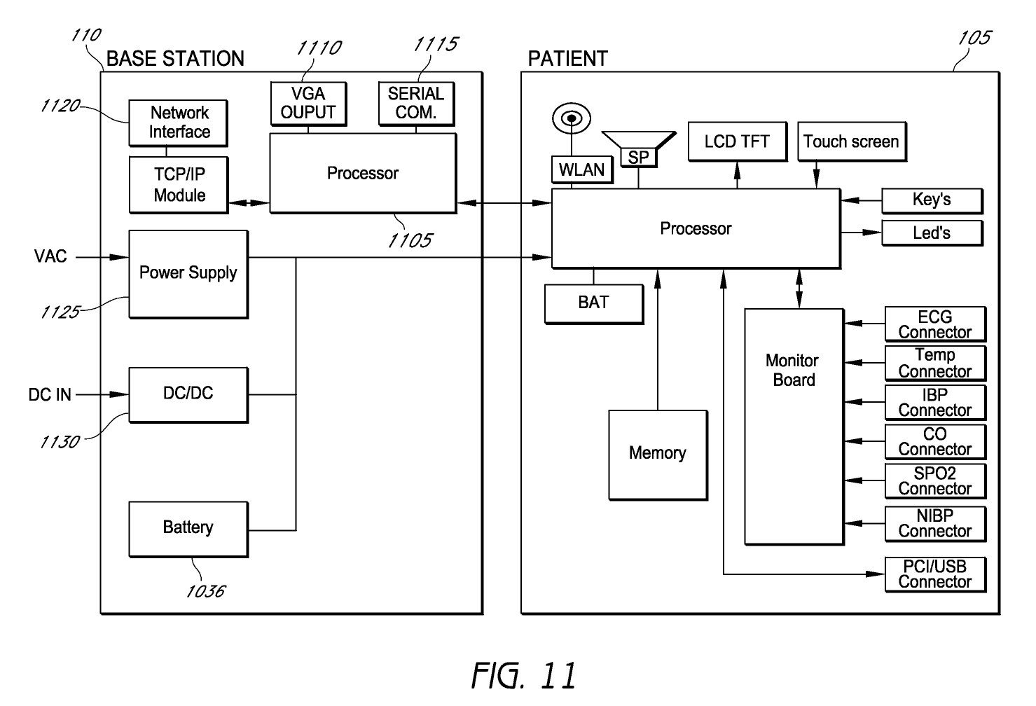

FIG. 11 illustrates a block diagram of the base station of FIG. 7. The base station 110 includes a processor 1105, memory, a display output 1110, serial port 1115, a network interface 1120, a power supply 1125, DC/DC converter 1130 and/or optional battery 1036. The base station can process display data from the patient monitor and output it to an external display and/or built-in display.

Furthermore, in certain embodiments, the systems and methods described herein can advantageously be implemented using computer software, hardware, firmware, or any combination of software, hardware, and firmware. In one embodiment, the system includes a number of software modules that comprise computer executable code for performing the functions described herein. In certain embodiments, the computer-executable code is executed on one or more general purpose computers or processors. However, a skilled artisan will appreciate, in light of this disclosure, that any module that can be implemented using software can also be implemented using a different combination of hardware, software or firmware. For example, such a module can be implemented completely in hardware using a combination of integrated circuits. Alternatively or additionally, such a module can be implemented completely or partially using specialized computers or processors designed to perform the particular functions described herein rather than by general purpose computers or processors.

Moreover, certain embodiments of the invention are described with reference to methods, apparatus (systems) and computer program products that can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the acts specified herein to transform data from a first state to a second state.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Various patient monitoring systems have been disclosed in detail in connection with various embodiments. These embodiments are disclosed by way of examples only and are not to limit the scope of the claims that follow. Indeed, the novel methods and systems described herein can be embodied in a variety of other forms; furthermore, various omissions, substitutions and changes in the form of the methods and systems described herein can be made without departing from the spirit of the inventions disclosed herein. The claims and their equivalents are intended to cover such forms or modifications as would fall within the scope and spirit of certain of the inventions disclosed herein. One of ordinary skill in the art will appreciate the many variations, modifications and combinations. For example, the various embodiments of the patient monitoring system can be used with sensors that can measure any type of physiological parameter. In various embodiments, the displays used can be any type of display, such as LCDs, CRTs, plasma, and/or the like. Further, any number of displays can be used as part of the patient monitoring system and multiple patient monitoring systems can be operated in tandem on the same patient.

What is claimed is:

1. A system for monitoring at least one physiological condition of a patient, the system comprising:
a physiological measurement device comprising a first display and a first processor, the first processor configured to process measurement data to determine values of one or more physiological parameters, the physiological measurement device providing monitoring of the one or more physiological parameters of a patient through communication with one or more sensors, the first display outputting a first display screen of values of the one or more physiological parameter, the physiological measurement device providing a video output signal to a second display in electrical communication with the physiological measurement device, the video output signal causing the second display to output a second display screen of values of the one or more physiological parameters; and
a controller, separately housed from said physiological measurement device, the controller in electrical communication with the physiological measurement device, the controller including at least one input for receiving a command from a user, the controller configured to transmit the received command to the physiological measurement device, the command causing the video output signal to change, the changed video output signal causing the second display output to change.

2. A system of claim 1, further comprising:
a docking station forming a patient monitoring system when combined with the physiological measurement device, the docking station configured to mate with the physiological measurement device when the physiological measurement device is operating in at least a docked mode, the docking station configured to receive the video output from the physiological measurement device and transmit the video output to the second display, the physiological measurement device in electrical communication with the docking station,
wherein the physiological measurement device is capable of operating in a portable mode, the portable mode providing portable monitoring as a standalone unit of the one or more physiological parameters of the patient, and capable of operating in the docked mode, the docked mode also providing monitoring of one or more physiological parameters of a patient.

3. The system of claim 1, wherein the change to the second display output comprises displaying at least one of a waveform, a value for a physiological parameter, and additional information about a monitored physiological parameter.

4. The system of claim 2, wherein the docking station comprises a built-in display.

5. The system of claim 1, further comprising a mount configured to attach to the second display, the mount configured to receive the controller.

6. The system of claim 5, the mount further comprising an adhesive layer for attaching the mount to the second display.

7. The system of claim 1, wherein the at least one input of the controller comprises a touch screen sensor on the second display.

8. The system of claim 1, wherein the physiological measurement device comprises a video output.

9. The system of claim 1, wherein the video output signal is transmitted through at least one of a VGA, DVI, HDMI, DisplayPort, WHDI, and WirelessHD connection.

10. The system of claim 1, wherein the values of the one or more physiological parameters comprise at least one of a numerical value and a waveform.

11. The system of claim 1, wherein the values of the one or more physiological parameters comprise one or more of blood oxygen content, respiratory gas, blood pressure, ECG, and pulse rate.

12. The system of claim 1, wherein the one or more sensors comprises a pulse oximetry sensor.

13. The system of claim 1, wherein the first display does not change when the second display changes.

14. A method for controlling the output of an external monitor, the method comprising:
monitoring one or more physiological parameters of a patient with one or more sensors in communication with a patient monitoring system;
transmitting one or more output signals comprising values of the one or more physiological parameters from the patient monitoring system to one or more external displays, a particular output signal causing a particular external display to generate a screen of the one or more physiological parameters;
receiving a command from a remote station; and
in response to the command, changing the particular output signal of the particular external display, causing the particular external display to generate a modified screen.

15. The method of claim 14, wherein the remote station is associated with the particular output signal.

16. The method of claim 14, wherein the modified screen comprises at least one of a waveform, a value for a physiological parameter, and additional information about a monitored physiological parameter.

17. The method of claim 14, wherein the patient monitoring system comprises:
a physiological measurement device capable of operating in a portable mode, the portable mode providing portable monitoring as a standalone unit of the one or more physiological parameters of the patient, and capable of operating in a docked mode, the docked mode also providing monitoring of one or more physiological parameters of a patient; and
a docking station forming a patient monitoring system when combined with the physiological measurement device, the docking station configured to mate with the physiological measurement device when the physiological measurement device is operating in at least a docked mode, the docking station configured to receive the one or more output signals from the physiological measurement device, the physiological measurement device in electrical communication with the docking station,

18. The method of claim 14, wherein the values of the one or more physiological parameters comprise one or more of blood oxygen content, respiratory gas, blood pressure, ECG, and pulse rate.

19. A remote station for controlling a display of a patient monitoring system, the remote station comprising:
a controller in electrical communication with a patient monitoring system, the controller including at least one input for receiving a command from a user, the controller configured to transmit the received command to the patient monitoring system, the command causing an output signal comprising one or more physiological parameters of a patient from the patient monitoring system to change, the changed output signal causing a display displaying a screen of the one or more physiological parameters to change; and
a mount for receiving a controller, the mount configured to attach to the display.

20. The remote station of claim 19 wherein the controller transmits the received commands to the patient monitoring system wirelessly.

21. The remote station of claim 19 wherein the controller transmits the received commands to the patient monitoring system through a cable.
